(19) United States
(12) Patent Application Publication
(10) Pub. No.: US 2024/0122861 A1
(43) Pub. Date: Apr. 18, 2024

(54) ELECTRORESPONSIVE BIOPOLYMER CAPSULES FOR ELECTRICALLY MEDIATED DELIVERY OF ACTIVES

(71) Applicant: University of Maryland, College Park, College Park, MD (US)

(72) Inventors: Srinivasa R. Raghavan, Columbia, MD (US); Ankit Gargava, Hillsboro, OR (US); Wenhao Xu, College Park, MD (US)

(21) Appl. No.: 18/489,408

(22) Filed: Oct. 18, 2023

Related U.S. Application Data
(60) Provisional application No. 63/380,002, filed on Oct. 18, 2022.

(51) Int. Cl. A61K 9/48; A61K 9/00; A61K 47/02; A61K 47/20; A61K 47/36; A61K 49/00

(52) U.S. Cl. CPC A61K 9/4816; A61K 9/0009; A61K 47/02; A61K 47/20; A61K 47/36; A61K 49/0047; A61K 49/0054; A61K 49/0093

(57) ABSTRACT

Surprisingly, electric fields can induce a dramatic response in soft materials made from nonconducting biopolymers. Capsules made from Alginate, Chitosan, and Gellan gum, all of which are charged polysaccharides and are biocompatible and biodegradable. Each capsule is formed by crosslinking biopolymer chains via physical (ionic/electrostatic) interactions. Under a DC electric field, the capsules rupture and disintegrate in a span of less than five minutes. The mechanism for the electroresponse is attributed to electrophoretic rearrangement of ions and/or polyelectrolyte chains in the capsule. Alginate capsules first swell anisotropically on their side closer to the anode (+ electrode). Cations migrate away from the anode, thereby lowering the crosslink density on that side. As further crosslinks are lost from the anode side, the capsule eventually breaks. A valve design utilizes an orifice that is blocked by a capsule and the valve is opened when the capsule is dislodged by the field.

### CROSS REFERENCE TO RELATED APPLICATIONS

This application claims priority under 35 U.S.C. § 119 to provisional patent application U.S. Ser. No. 63/380,002, filed Oct. 18, 2022. The provisional patent application is herein incorporated by reference in its entirety, including without limitation, the specification, claims, and abstract, as well as any figures, tables, appendices, or drawings thereof.

### STATEMENT REGARDING FEDERALLY SPONSORED RESEARCH OR DEVELOPMENT

This invention was made with government support under W911NF1820170 awarded by the Department of the Army, Army Research Office. The government has certain rights in the invention.

### TECHNICAL FIELD

The present disclosure relates generally to electroresponsive biopolymer capsules that are capable of delivering encapsulated actives. The electroresponsive biopolymer capsules have applications in at least the pharmaceutical, biomedical, and electronic industries.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Soft materials filled with water are widely encountered in biomedical and consumer products. Two classes of such materials are hydrogels electroresponsive biopolymer capsules for delivery of encapsulated actives and capsules. A hydrogel is a water-filled network of polymer chains, with the network being formed by either covalent or physical crosslinks between the chains. A capsule is a spherical structure in which the core is either an aqueous solution or gel whereas the shell is a distinct layer. In some cases, the shell can have the same chemistry as the core but a higher degree of crosslinking. In other cases, the shell can be a coacervate or even a covalently crosslinked layer whereas the core can be physically crosslinked. Both gels and capsules find use in biomedical applications such as drug delivery and tissue engineering, as well as in soft robotics and wearable devices.

A recurring theme has been to make these gels and capsules responsive to external stimuli such as solution pH, salt concentration, temperature, light (at various wavelengths), magnetic fields, and electric fields. Among the stimuli of interest, an emerging one is the electric field, which is particularly attractive because it can be easily turned on and off at a particular location (i.e., with spatial precision) as well as at a precise time (i.e., with temporal precision). Other stimuli such as pH, ionic strength, and temperature cannot achieve the same level of spatial or temporal precision. Light can do so under some cases, but light gets attenuated as it passes through many materials. Moreover, light sources are bulky whereas electric fields can be applied with just a battery (DC) or by connecting to the commercial AC electric supply. In turn, electrical stimuli can be exploited even in portable devices that can be controlled wirelessly and from remote locations, e.g., through the Internet (via Bluetooth).

Electroresponsive soft materials have typically been made using conductive polymers (CPs), such as polypyrroles and polythiophenes, where the charge carriers are electrons. For example, drugs have been chemically conjugated to such CPs, and upon applying an electric current, the release of these drugs from the CPs has been triggered. Alternatively, instead of CPs, electronic conductivity has been imparted to soft materials by adding conductive nanoparticles such as carbon nanotubes (CNTs). For instance, capsules were created with CNTs in their shells and found that the capsules became more permeable under an electric field. However, materials such as CPs and CNT-based composites have their limitations because they are either expensive or difficult to synthesize and are generally not biodegradable or biocompatible. Note also that, for a response to occur in an electronically conductive material, it must be in direct contact with an electrode (i.e., the source of current).

Thus, there exists a need in the art for cost-effective, easy to synthesize electroresponsive biopolymer capsules that are capable of delivering encapsulated actives.

An alternative to electrons is to rely on ions as the charge carriers. Polyelectrolytes, i.e., polymers with ionizable backbones, are widely available and are biocompatible. Hydrogels based on polyelectrolytes have been investigated in electric fields. The hydrogels can be placed in solution either with or without direct contact with the electrodes before applying a current. In some studies, bulk gels have been reported to shrink (or swell) under a field. If such shrinking occurs non-uniformly, the gel can be also made to bend, and this bending can further be transduced into a slithering motion of the gel. Several others have also reported that gel films can be eroded by applying a field (all these films were in contact with one electrode. Such erosion of gel films has been used to deliver drugs entrapped in the films. In addition, electrical response has also been reported for certain nanoscale polymer vesicles (formed by the self-assembly of special block copolymers or homopolymers) by exploiting their sensitivity to redox conditions.

There also exists a need in the art to investigate spherical gels or capsules made from polyelectrolytes and significant transformations to such capsules by electric fields.

### SUMMARY

Two types of capsules that have been extensively studied by the present inventors are based on the common biopolymers (polysaccharides) Alginate and Chitosan. See e.g.: Lee, et al., “Biopolymer capsules bearing polydiacetylenic vesicles as colorimetric sensors of pH and temperature.” Soft Matter 2011, 7, 3273-3276; Dowling, et al., “Self-destructing ‘mothership’ capsules for timed release of encapsulated contents.” Langmuir 2013, 29, 7993-7998; Ghaffarian, et al., “Chitosan-Alginate microcapsules provide gastric protection and intestinal release of ICAM-1-targeting nanocarriers, enabling GI targeting in vivo.” Adv. Funct. Mater. 2016, 26, 3382-3393; and Lu et al. “A new design for an artificial cell: Polymer microcapsules with addressable inner compartments that can harbor biomolecules, colloids or microbial species.” Chem. Sci. 2017, 8, 6893-6903. Each of the aforementioned publications are hereby incorporated by reference in their entireties herein.

In the case of a gel, it can be induced to swell or shrink in response to the above stimuli. See e.g., Gargava, et al., “Smart hydrogel-based valves inspired by the stomata in plants.” ACS Appl. Mater. Interfaces 2016, 8, 18430-18438. Alternatively, a bulk gel can be actuated to change its shape: for example, a flat sheet can fold into a pancake or tube in response to the same stimuli. See e.g., Athas et al., “Cation-induced folding of alginate-bearing bilayer gels: an unusual example of spontaneous folding along the long axis.” Soft Matter 2018, 14, 2735-2743. In the case of capsules, responses that can be induced include (i) capsule inflation and bursting, see e.g., DeMella et al., “Catalyst-loaded capsules that spontaneously inflate and violently eject their core.” Langmuir 2019, 35, 13718-13726; (ii) autonomous capsule motion, see e.g., Lu et al., “Catalytic propulsion and magnetic steering of soft, patchy microcapsules: Ability to pick-up and drop-off microscale cargo.” ACS Appl. Mater. Interfaces 2016, 8, 15676-15683; (iii) capsule color changes; or (iv) an abrupt change in capsule permeability, see e.g., Zarket et al., “Onion-like multilayered polymer capsules synthesized by a bioinspired inside-out technique.” Nat. Commun. 2017, 8, 193. Each of the aforementioned publications are hereby incorporated by reference in their entireties herein.

The use of electric fields to stimulate the delivery of drugs or other active ingredients is of great interest for wearable electronics and other applications. Most attempts at electrically induced delivery with soft materials in water have focused on electronically conducting polymers (e.g., polypyrroles) or conductive nanocomposites (e.g., polymers with carbon nanotubes). Here, electrical responses are induced even in structures made from nonconducting biopolymers that are widely available, biocompatible, and biodegradable. For example, spherical capsules can be created from the anionic polysaccharide Alginate by crosslinking with cations like Ca²⁺ or Cu²⁺. When these capsules are placed in aqueous solution and subjected to a DC electric field of about 8 V/cm, they deform within a couple of minutes and then burst and disintegrate into pieces within about five minutes (5 min). Capsules across a range of length scales such as two hundred micrometers to two centimeters (200 μm to 2 cm) respond in the above manner, and the electroresponse persists even if the capsules are embedded in a nonionic gel matrix. The electroresponse is due to electrophoretic migration of charged species (ions and/or polyelectrolyte chain-segments) within (or out of) the capsules. In an Alginate capsule, the cations are induced to migrate away from the positive electrode, which creates a weakly-crosslinked region of the capsule that swells appreciably. This anisotropic swelling continues until the capsule eventually bursts. Electroresponsive capsules have applications that highlight the spatial and temporal accuracy possible with an electrical stimulus. The bursting of capsules can be used to release solutes loaded inside these structures. Also, even the deformation of intact capsules can be used to create electrically actuatable valves, where a liquid flows out through the valve only when a capsule plug is dislodged. Examples of the present disclosure involve and include electrically induced bursting of aqueous capsules made from biopolymers based on ‘switching on’ the release of payloads.

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of the present disclosure to place soft materials in water and to allow for those soft materials to respond to an electric field. Further efforts are undertaken to determine what types of materials comprise said soft materials. For example, capsules (with liquid or gelled cores) of anionic Alginate and the cationic Chitosan polymers, formed by noncovalent interactions, can be placed in water and a moderate DC electric field (˜10 V/cm) is applied using remote electrodes (i.e., the electrodes do not touch the capsules). The capsules deform or swell anisotropically within a minute and then rapidly burst within about five minutes (5 min), thereby releasing their internal contents.

It is still yet a further object, feature, and/or advantage of the present disclosure to provide biodegradable and/or biocompatible electroresponsive biopolymer capsules. For example, a significant electroresponse is indeed possible in spherical gels and capsules made from common biopolyelectrolytes—including the anionic Alginate and the cationic Chitosan—which are widely used in biomedical applications.

It is still yet a further object, feature, and/or advantage of the present disclosure to determine an optimal size for said capsules. Capsules across a range of length scales: two hundred micrometers to two centimeters (200 μm to 2 cm) can deform or swell anisotropically quickly, thereby releasing their internal contents.

It is still yet a further object, feature, and/or advantage of the present disclosure to tune the electroresponse of the capsules by varying the field strength as well as the capsule composition. A mechanism based on electrophoretic migration of charged species (ions and/or chain-segments) can be employed within or out of the capsules.

It is still yet a further object, feature, and/or advantage of the present disclosure to deliver encapsulated actives without requiring direct contact with an electrode, using the electroresponsive biopolymer capsules. The encapsulated actives that are delivered can include a wide variety of molecular weights, such as small matter with a molecular weight of ˜5000 g/mol, and larger matter with a molecular weight of ˜40,000 g/mol (e.g. proteins). The encapsulated actives can be and/or can be embedded within biologics. The ability to “deliver on demand” several types of encapsulated actives is a great benefit that is not solved by others in the art.

It is still yet a further object, feature, and/or advantage of the present disclosure to provide the ability to shape the soft structures that form the electroresponsive biopolymer capsules. For example, the electroresponsive biopolymer capsules can be spherical prior to be electrically actuated.

It is still yet a further object, feature, and/or advantage of the present disclosure to determine and/or control ideal times for deformation of the electroresponsive biopolymer capsules. Not all of the electroresponsive biopolymer capsules placed in an electrified aqueous solution will break at the same time under identical or even near identical conditions. Rather, it appears that while there is consistency in that the electroresponsive biopolymer capsules will all be destroyed within a small time (e.g., ˜1 min, ˜5 min, ˜10 min, etc.), there will is some variance as to the exact time of when, for example, encapsulated actives would be released from therewithin. Furthermore, it appears (i) the distance from the electroresponsive biopolymer capsules to the electrodes; (ii) the number of electroresponsive biopolymer capsules placed in the aqueous solution; and (iii) the temperature of the aqueous solution and/or electroresponsive biopolymer capsules, have little to no effect as to the time of deformation/rupture. The largest determinant of this time appears to be the composition of the electroresponsive biopolymer capsules, discussed extensively herein.

It is still yet a further object, feature, and/or advantage of the present disclosure to require only a small amount of electrical energy in order to actuate the deformation and/or collapse of the electroresponsive biopolymer capsules. For example, ˜5V is a most preferred power requirement, while ˜10V is a much preferred power requirement, and ˜15V is an acceptable power requirement. This means, as an example, devices employing 3AAA batteries could be used to cause the rupture of said electroresponsive biopolymer capsules. Said devices could even instruct the rupture of said electroresponsive biopolymer capsules in a wireless manner.

The electroresponsive biopolymer capsules disclosed herein can be used in a wide variety of applications. Electrically induced bursting of capsules could be used for the release of encapsulated payloads such as drugs, perfumes, or agrochemicals. Also, the same capsules could be used to create electroactuated valves, which open to allow liquid flow only when an electric field is switched on.

The electroresponsive biopolymer capsules disclosed herein can be incorporated into arrays of valves, other systems, and/or kits which accomplish some or all of the previously stated objectives.

Methods can be practiced which facilitate use, manufacture, synthetization, assembly, maintenance, and repair of electroresponsive biopolymer capsules which accomplish some or all of the previously stated objectives.

According to some aspects of the present disclosure, a system for delivering encapsulated actives comprises an electroresponsive biopolymer capsule that has a cationic component and an anionic component that complements the cationic component and a cell that includes an aqueous solution and a pair of electrodes at least partially submerged in the aqueous solution. Electrolysis of the aqueous solution causes an electrophoretic rearrangement of ions or polyelectrolyte chains in the electroresponsive biopolymer capsule, which deforms the electroresponsive biopolymer capsule. Optionally, the electrodes can comprise graphite slabs. The electrolysis process can be analogized to electrifying an bag that holds a liquid (and the biopolymer).

According to some additional aspects of the present disclosure, the electroresponsive biopolymer capsule comprises a polymer ion capsule. For example, the cationic component can comprise Cu²⁺ or Ca²⁺ multivalent cations, the anionic component can comprise an Alginate, and/or the polymer ion capsule can be embedded in an Agarose gel.

According to some additional aspects of the present disclosure, the electroresponsive biopolymer capsule comprises an inner core and an outer shell. The outer shell should be distinct in composition form the core of the inner shell.

According to some additional aspects of the present disclosure, the electroresponsive biopolymer capsule comprises a polymer-surfactant capsule. For example, the cationic component can comprise a Chitosan biopolymer and/or the anionic component can comprise a sodium dodecyl benzene sulfonate (SDBS) surfactant.

According to some additional aspects of the present disclosure, the electroresponsive biopolymer capsule comprises a polymer-polymer capsule. For example, the cationic component can comprise a Chitosan biopolymer and/or the anionic component comprises a nonconducting biopolymer: Gellan gum.

According to some additional aspects of the present disclosure, the electroresponsive biopolymer capsule further comprises carbon black (CB) particles or fluorescent polystyrene latex particles. An inverted optical microscope that detects fluorescence can detect green-fluorescent polystyrene latex (GFPL) nanoparticles and therefore monitor whether the capsules have been ruptured.

According to some additional aspects of the present disclosure, the system further comprises a plurality of electroresponsive biopolymer capsules that are configured to function as a plurality of independently actuatable valves. The plurality of electroresponsive biopolymer capsules may or may not include the aforementioned electroresponsive biopolymer capsule unless otherwise noted.

According to some additional aspects of the present disclosure, the aqueous solution can be a NaCl solution. The aqueous solution can also be water.

According to some other aspects of the present disclosure, an electroresponsive biopolymer capsule comprises a cationic component; an anionic component that complements the cationic component; and an electrophoretic rearrangement of ions or polyelectrolyte chains that when electrically actuated causes the electroresponsive biopolymer capsule to rupture.

According to some other aspects of the present disclosure, a method for delivering encapsulated actives comprising loading an electroresponsive biopolymer capsule with encapsulated actives; and rupturing the electroresponsive biopolymer capsule as a result of a applying a direct current (DC) electric field to an electrophoretic rearrangement of ions or polyelectrolyte chains in the electroresponsive biopolymer capsule, thereby causing the encapsulated actives to be released from the electroresponsive biopolymer capsule.

According to some additional aspects of the present disclosure, the rupturing of the electroresponsive biopolymer capsule occurs regardless of whether there is a change in temperature in the system and/or the rupturing of the electroresponsive biopolymer capsule occurs without directly contacting the capsule.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIGS. 1A-1C show various biopolymer capsules that are created by combining an anionic or cationic polysaccharide with moieties of opposite charge. FIG. 1A shows a polymer-ion capsule formed by crosslinking anionic Alginate with multivalent cations (Cu2+ or Ca²⁺). FIG. 1B shows a polymer-surfactant capsule formed by combining cationic Chitosan with the anionic surfactant: sodium dodecyl benzene sulfonate (“SDBS”). FIG. 1C shows a polymer-polymer capsule formed by combining anionic Gellan gum with cationic Chitosan. In all cases throughout FIGS. 1A-1C, a solution of the polymer listed on the top is introduced as droplets into a reservoir containing the moiety listed on the bottom to form the capsules.

FIG. 2 shows a setup used to study the electrical response of capsules. Capsules are placed in a rectangular cell containing an aqueous solution. For example, the aqueous solution can be ten millimolar (10 mM) NaCl. The cell is flanked by two graphite electrodes, which are connected to the positive (+) and negative (−) terminals of a DC power source. The cell is placed on an inverted microscope for close observation of the capsules in the presence of the field.

FIGS. 3A-3B show electrical bursting of Alginate-Cu²⁺ microcapsules over time. FIG. 3A shows bare microcapsule (with no internal payload). FIG. 3B shows a microcapsule with carbon black (CB) nanoparticles. The CB helps in visualizing the rupture. For both FIGS. 3A-3B, the microcapsules were placed in ten millimolar (10 mM) NaCl solution and a DC voltage of fifteen volts (15 V) is applied. Images are shown at different time points. For both FIGS. 3A-3B, the microcapsules swelled on the side closer to the positive electrode before rupturing on that side. Scale bars: 100 μm.

FIGS. 4A-4B show electrical bursting of two microcapsule types over time. FIG. 4A shows Chitosan-SDBS with green-fluorescent polystyrene latex (GFPL) nanoparticles. FIG. 4B shows Gellan-Chitosan with CB nanoparticles. For both FIGS. 4A-4B, the microcapsules are placed in ten millimolar (10 mM) NaCl and fifteen volt direct current (15 V DC) is applied. Images are shown at different time points. For both FIGS. 4A-4B, the microcapsules ruptured on the side closer to the positive electrode. Scale bars: 100 μm.

FIG. 5 shows electrical bursting of a batch of Alginate-Cu²⁺ microcapsules. The microcapsules were placed in ten millimolar (10 mM) NaCl solution and fifteen volt direct current (15 V DC) is applied. Images of several microcapsules (all with CB) are shown at different time points. All microcapsules swelled on one side and ruptured, as shown previously in FIGS. 3A-3B. Scale bars: 300 μm.

FIGS. 6A-6C show effects of different variables on the electrical rupture of Alginate-Cu²⁺ microcapsules. Microcapsules (with CB) are placed in ten millimolar (10 mM) NaCl and a DC voltage is applied. The breaking time is the time at which the capsule first shows a rupture (as seen from the release of CB). FIG. 6A emphasizes the effect of varying the voltage (all capsules made by adding ten percent (2%) Alginate to eight percent (8%) Cu²⁺ and incubating for two minutes: 2 min). FIG. 6B emphasizes the effect of incubation time (all capsules made by adding two percent (2%) Alginate to eight percent (8%) Cu²⁺ and incubated for different lengths of time). The longer the incubation, the stronger the capsule. FIG. 6C emphasizes the effect of Cu²⁺ concentration (all capsules made by adding 2% Alginate to varying Cu²⁺ and incubating for 2 min). The higher the Cu²⁺, the more crosslinked the capsule. Lines in all plots throughout FIGS. 6A-6C are to guide the eye. For each data point, at least three samples were studied, and the data shown are the mean values. Error bars correspond to standard deviations.

FIGS. 7A-7C show electrical bursting of various macrocapsules over time. FIG. 7A shows Alginate-Cu²⁺ with carbon black (CB); FIG. 7B shows Chitosan-SDBS; and FIG. 7C shows Gellan-Chitosan with CB. The CB nanoparticles in the capsule core help to visualize the rupture. In all cases, the capsules are placed in a ten millimolar (10 mM) NaCl solution and a DC voltage of fifteen volts (15 V) is applied at t=0. Images are shown at different time points. All the capsules rupture over time, but do not completely disintegrate. Scale bars: 3 mm.

FIG. 8 show breaking times of Alginate-Mⁿ⁺ macrocapsules formed with different cations. Capsules are placed in ten millimolar (10 mM) NaCl and fifteen volts direct current (15 V DC) is applied. The breaking time is the time at which the capsule first shows a rupture. All capsules were formed by dropping two percent (2%) Alginate 104 into eight percent (8%) solutions 102 of the respective cations and incubating for two minutes (2 min). For each case, at least three samples were studied and the averages are plotted. Error bars correspond to standard deviations.

FIGS. 9A-9B show a mechanism for electrical bursting of Alginate-Cu²⁺ capsules. As shown in FIG. 9A, the electrophoresis of charged species, in this case Cu2+ cations, migrate towards the −ve electrode. Such migration within the capsule will create zones that are enriched or depleted in these crosslinking ions. Some cations are also expected to be released out of the capsule. FIG. 9B schematically shows changes in a single capsule. The side near the +ve electrode will have a lower Cu²⁺ concentration, and the depletion of crosslinks (increase in mesh size) will induce this side to swell. Such swelling will continue until this side of the capsule eventually ruptures.

FIGS. 10A-10B show evidence for proposed capsule-bursting mechanism from colorimetry. The dye eriochrome black T (EBT) is a known colorimetric indicator of Ca²⁺. FIG. 10A shows standard solutions containing EBT and increasing amounts of Ca²⁺ show a transition from blue to violet to red. FIG. 10B shows the EBT assay is repeated with the solution around Alginate-Ca²⁺ macrocapsules. In the control case (Tube 1), the capsules are in solution with no voltage applied—the Ca2+ concentration in the solution is then very low. If a low voltage (6 V, Tube 2 or 7 V, Tube 3) is applied for ten minutes (10 min), the capsules do not break, and a slightly higher Ca²⁺ is detected in the solution. If a voltage of fifteen volts (15 V) is applied for ten minutes (10 min), the capsules still do not break, but appreciable Ca²⁺ can be detected in the external solution. This result supports the postulated mechanism involving Ca²⁺ electrophoresis within the capsules, which is depicted in FIGS. 9A-9B and discussed in the Detailed Description, infra.

FIGS. 11A-11B show an electro-actuated fluid valve. FIG. 11A shows an Alginate capsule is wedged in at the end of a plastic transfer pipette to block the gravity-driven downward flow of a dye solution. The assembly is placed between two electrodes and the closed end is immersed in 10 mM NaCl. FIG. 11B shows when the electric field (15 V DC) is applied, the capsule deforms within 1 min, thereby opening the valve. Thereafter, within 2 min, the capsule gets dislodged. As a result, the valve is fully opened, allowing the dye solution to flow out.

FIGS. 12A-12D show sequential actuation of three valves by spatial and temporal control of the electric field. Specifically: in FIG. 12A all three valves are closed at t=0; in FIG. 12B dye flows out of valve 3; in FIG. 12C, dye flows out of valve 1; and in FIG. 12D, dye flows out of valve 2. Microcapsules containing each microbe are made first. These are mixed with Alginate and used as a feed for the MCCs. The feed is flowed through a 400 μm capillary and droplets are sheared off the capillary tip by pulses of nitrogen gas. The droplets are collected in the reservoir, where they are converted to MCCs due to crosslinking of the Alginate by Ca²⁺ and Chitosan.

An artisan of ordinary skill in the art need not view, within isolated figure(s), the near infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

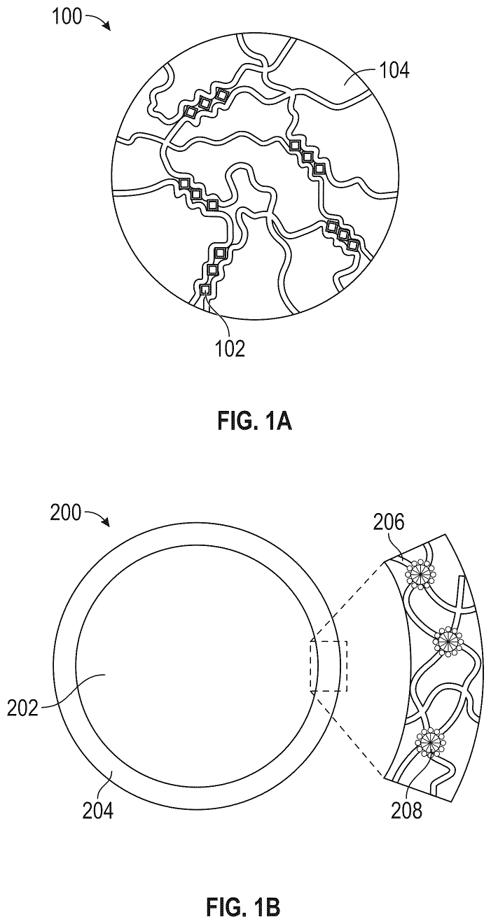

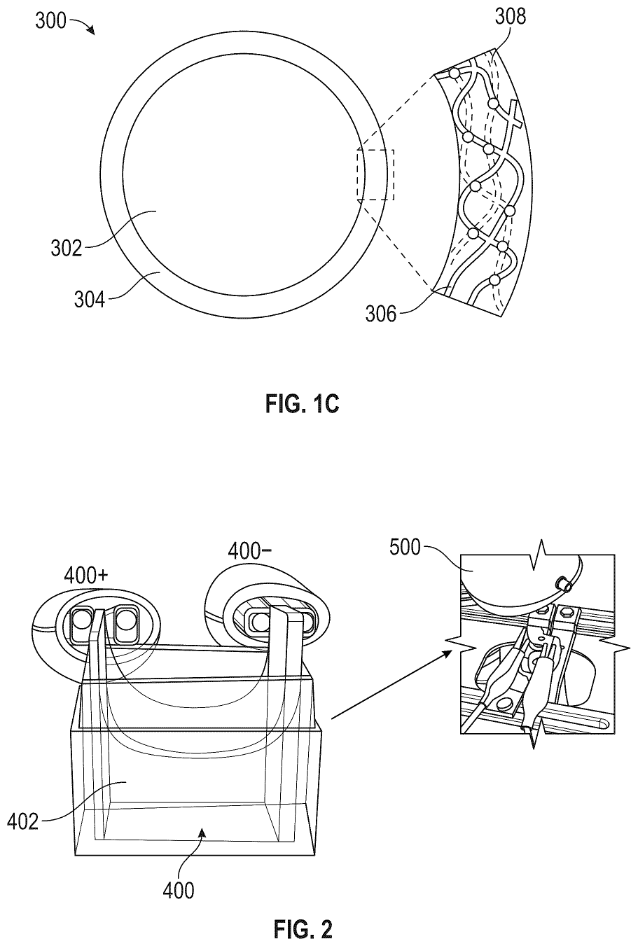

Three types of spherical particles made from biopolymers, all of which can be referred to as ‘capsules’, are shown throughout FIGS. 1A-1C. In each case, the particle core and shell are different in some way.

The first kind of capsule shown in FIG. 1A is a polymer ion capsule 100 (e.g., Aliginate-Cu²⁺/Ca²⁺). The particular polymer ion capsule 100 shown in FIG. 1A is made by dropping an Alginate solution 104 into a reservoir solution of multivalent cations 102 such as Ca²⁺, Cu²⁺, Zn²⁺, or Fe²⁺. Alginate chains 104 become crosslinked by the cations 102 into a gel-network. Note that the Alginate chains 104 are bound by cations 102 along ‘egg-box’ junctions 106. If the droplets are incubated in the reservoir for a long time, e.g., approximately ten minutes (˜10 min), the crosslinking will be uniform throughout the droplet. For shorter incubation times, e.g., approximately five minutes (˜5 min), the droplet core will be crosslinked less than the periphery, resulting in a core-shell structure, i.e., in the polymer ion capsule 100. Alginate capsules 100 (also sometimes termed ‘beads’, or simply ‘gels’) and are usually formed with calcium (Ca²⁺). Here, copper (Cu²⁺) is used as the crosslinking cation because capsules of the latter are more stable and robust than those of the former.

The second kind of capsule shown in FIG. 1B is a polymer-surfactant capsule 200 (e.g., Chitosan-SDBS). The particular polymer-surfactant capsule 200 shown in FIG. 1B is made by adding droplets of the cationic biopolymer Chitosan 206 into a solution of the anionic surfactant sodium dodecyl benzene sulfonate (SDBS) 208. Polymer-surfactant capsules 200 with a liquid core 202 and a shell 204 are formed by electrostatic complexation (akin to coacervation) of the oppositely charged polymer and surfactant.

The third kind of capsule shown in FIG. 1C is a polymer-polymer capsule 300 (e.g., Gellan-Chitosan). The polymer-polymer capsule 300 is made by adding another anionic biopolymer Gellan gum 208 into a solution of the cationic polymer Chitosan 206. Capsules with a liquid core 202 and a shell 204 are formed featuring a coacervate of the oppositely charged polymers.

The capsules 100, 200, 300 in FIGS. 1A-1C have been deliberately chosen as representative examples for three distinct modes of physical crosslinking, involving: (a) a polymer and multivalent ions of opposite charge; (b) a polymer and an oppositely charged surfactant; and (c) two oppositely charged polymers.

The above capsules 100, 200, 300 were prepared over a range of sizes, which for simplicity are classified as being in the macroscale (greater than one millimeter in diameter: >1 mm) or the microscale (less than one millimeter in diameter <1 mm). In the case of macroscale capsules, droplet sizes were controlled using plastic pipettes or syringe needles with different gauges. To prepare microscale capsules, the microfluidic setup described in Ghaffarian et al. and Lu et al. (see the preceding citations and incorporations by reference, supra), were used. A key distinguishing feature of this setup is the use of gas as the continuous phase instead of oil. Pulses of compressed air or nitrogen gas are used to shear off aqueous microdroplets from a capillary tip. The microdroplets are then added to a reservoir solution, as before, where they are converted to microcapsules. The microcapsule size is controlled by the feed flow rate and the frequency of gas pulses.

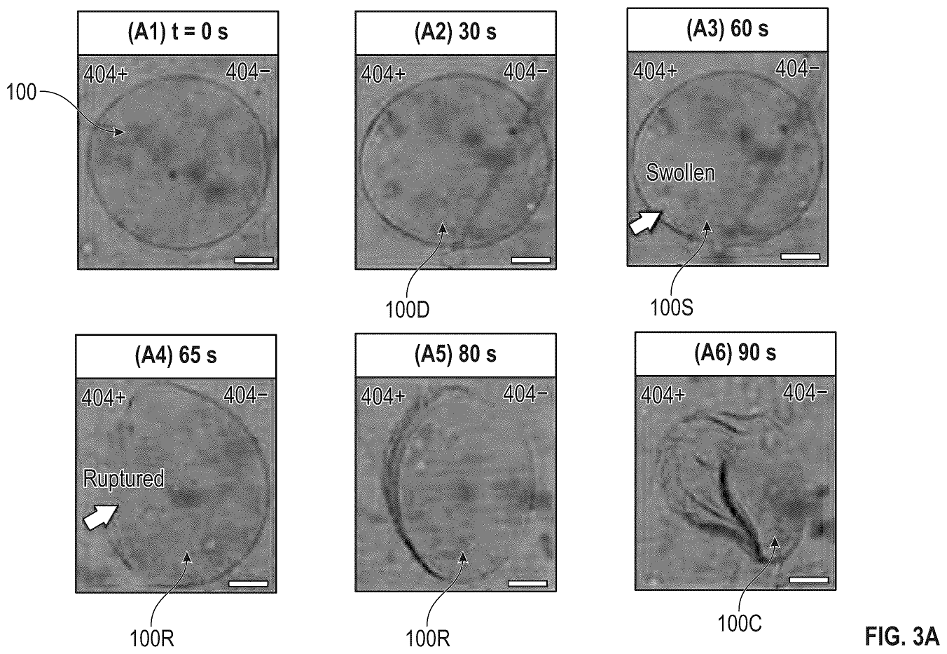

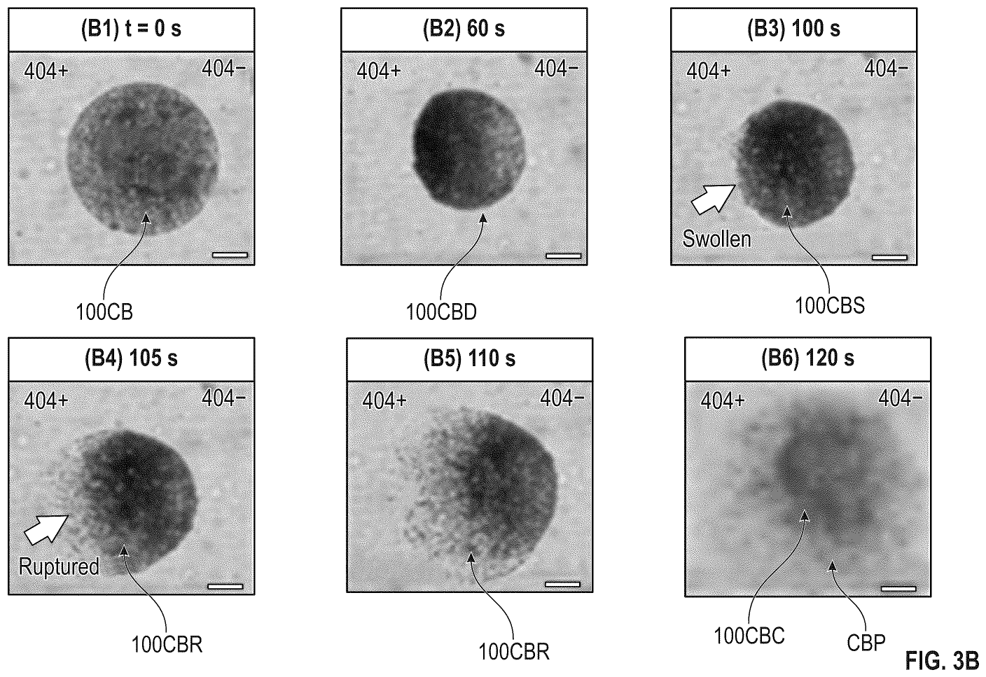

An electric field was applied to the capsules 100, 200, 300 using the rectangular cell 400 shown in FIG. 2. The capsule 100, 200, 300 is placed in an aqueous solution 402 and graphite electrodes 400+, 400− are placed on either side, with the anode 400+ (positive electrode) on the left and the cathode 400− (negative electrode) on the right. This orientation of the capsule relative to the electrodes will become important in analyzing the results. FIG. 3A shows the first set of results, which are for Cu²⁺-crosslinked Alginate microcapsules 100 (two hundred micrometer radius: 200 μm) in a ten millimolar (10 mM) NaCl solution. A voltage of fifteen volts (15 V) at a field strength of eight volts per centimeter (8 V/cm) is applied across the test cell 400 at t=0, and the microcapsules are observed through an inverted optical microscope 500. The images in FIGS. 3A-3B are for empty microcapsules whereas those in FIG. 3B are for microcapsules with 0.5% of carbon black (CB) nanoparticles dispersed in their core. The CB can be considered a model payload and their presence aids in visualization. As long as the capsule stays intact, the CB remains sequestered in the core. Both sets of images show preferential swelling of the microcapsule on its left side (nearer the positive electrode 404+) (Photos A3 and B3) after about a minute of applying the field. The swelling can be clearly seen in Photo B3 because the CB in the microcapsule gets diluted as the left side swells (i.e., this side looks more transparent). Once the swelling starts, it continues for the next ten to twenty seconds (10 to 20 s) until the left side of the microcapsule suffers a rupture (Photos A4 and B4). The final collapsed microcapsule looks like a deflated balloon in Photo A6. In Photo B6, the rupture of the microcapsule induces the payload (CB nanoparticles) to burst out and spread all over the microscopic field of view.

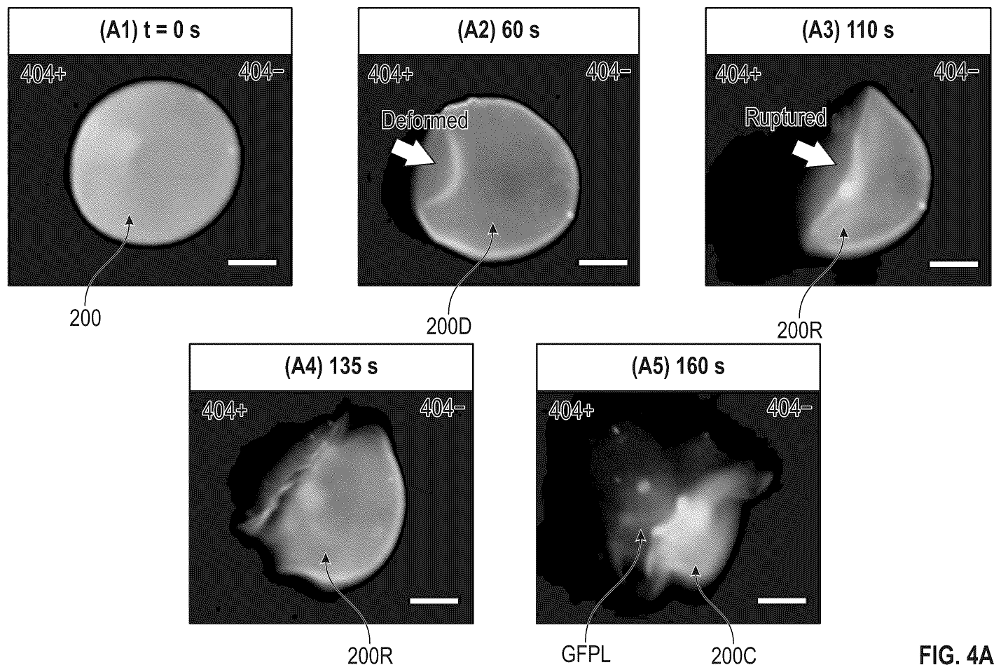

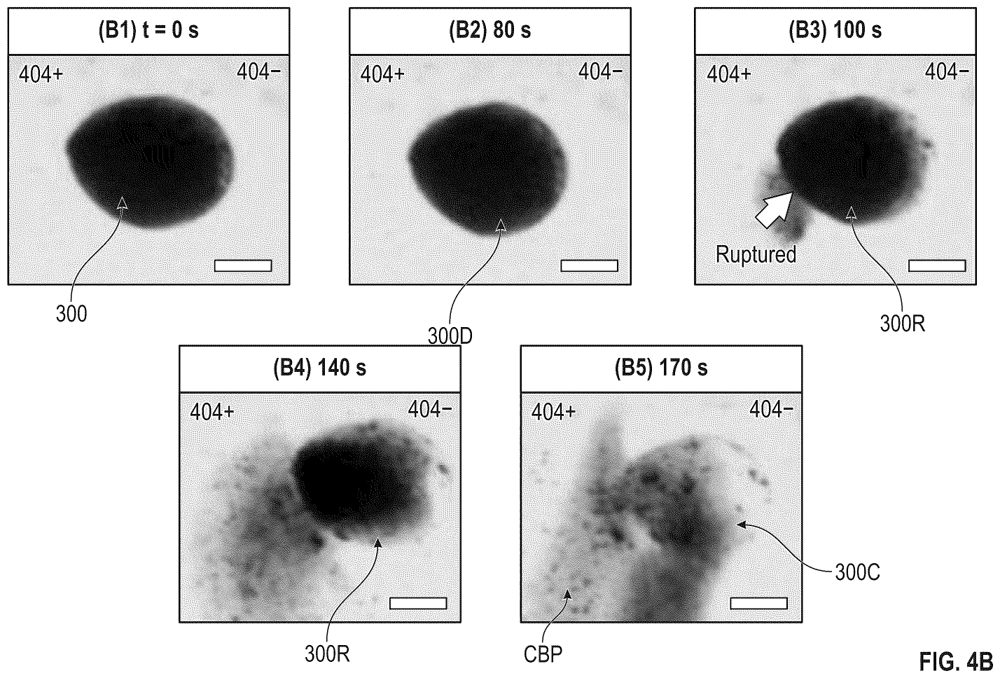

FIG. 4A shows the response of a Chitosan-SDBS microcapsule 200 with a two hundred micrometer radius (200 μm) to the same fifteen volts (15 V) voltage. The microcapsule contains 0.5% green-fluorescent microparticles dispersed in its core, and the images are taken with a fluorescence microscope 500. In this case, around the sixty seconds (60 s) mark (Photo A2), the left (+) side of the capsule appears to bend and fold inward. Thereafter, at approximately one hundred ten seconds (˜110 s) (Photo A3), this side breaks and the contents then spill out into the solution. Next, FIG. 4B shows a Gellan-Chitosan microcapsule 300 with a radius of two hundred micrometer radius (200 μm) with 0.5% CB nanoparticles. When subjected to a fifteen volts (15 V) field, a break in the left (+) side of the capsule shell is first seen at around the one hundred seconds (100 s) mark (Photo B3). This break grows over the next sixty seconds (60 s) and the capsule completely disintegrates. In the process, the CB nanoparticles again spill out of the capsule and spread all over the solution. Thus, the net result is the same for all three kinds of capsules studied in FIGS. 3A-3B and FIGS. 4A-4B—they all burst apart within a couple of minutes of switching on the electric field.

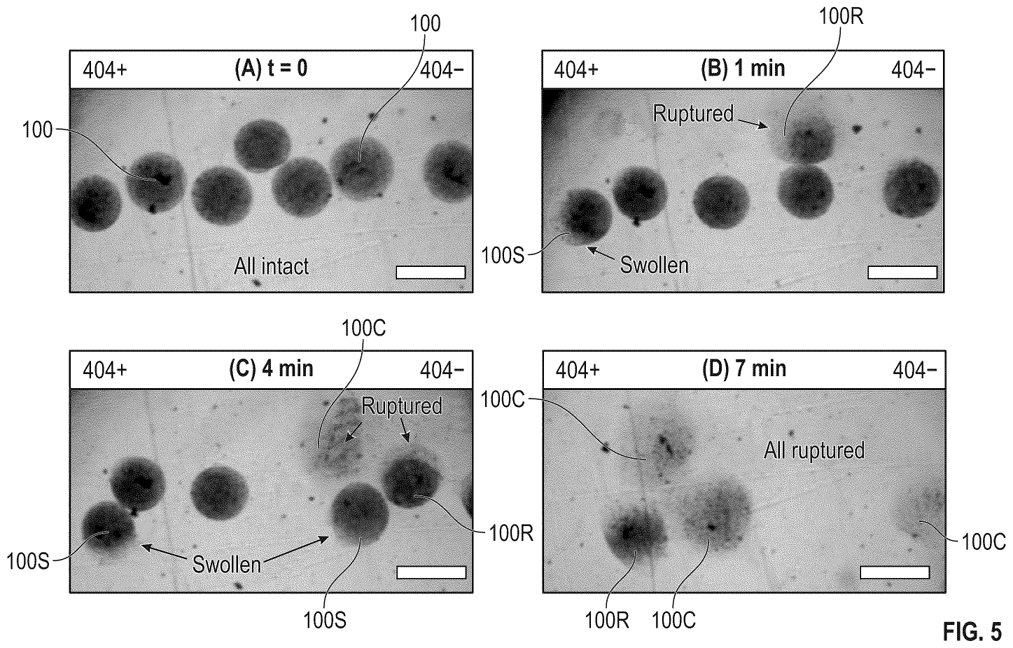

Electrically induced disintegration thus appears to be a widespread effect in capsules assembled by physical interactions. To show the generality of this phenomenon, the above experiments were repeated with multiple capsules in a couple of different ways. First, a number of Alginate-Cu²⁺ microcapsules 100 were placed in a millimolar (10 mM) NaCl solution 402 and applied the fifteen volts (15 V) field. As shown in FIG. 5, all the capsules swell on their side closer to the positive electrode 404+ and eventually break at this side within seven minutes (7 min). The capsules do not all break at the same instant of time, and there is no particular pattern or order to their breaking (e.g., distance to either electrode is not a factor). This was verified through different trials similar to the one shown in FIG. 5. In another variation, multiple capsules were placed in a gel before applying the field. For this, Alginate-Cu²⁺ microcapsules 100 were combined with a hot solution of the biopolymer Agarose and then cooled. The result was an Agarose gel with the capsules entrapped in the gel. The gel was cut into a one-centimeter (1-cm) cube, which was placed in a ten millimolar (10 mM) NaCl solution. When the fifteen volts (15 V) field was applied, all the capsules still broke within about seven minutes (7 min) in the same manner as in FIG. 5. Because capsule breakage is unaffected by the distance to either electrode, we conclude that electrochemical reactions at the electrodes are unlikely to be responsible for the phenomenon.

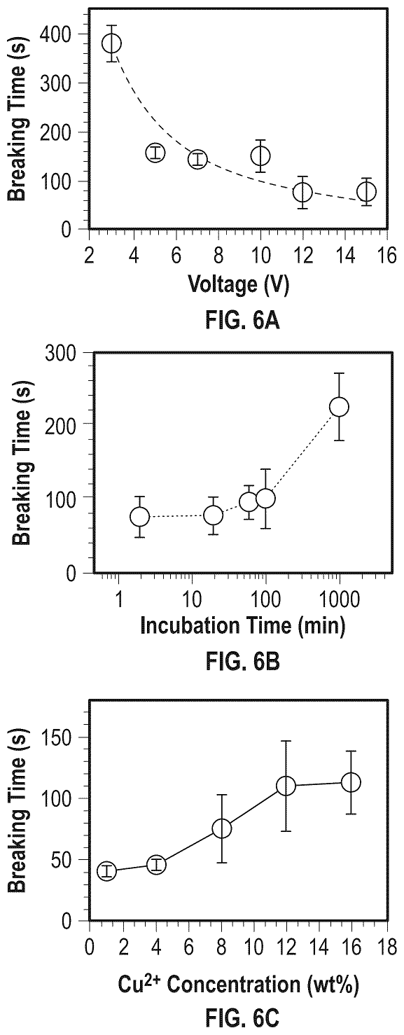

One can estimate the breaking time of a microcapsule, i.e., the time at which a rupture is first detected by optical microscopy. The effects of different variables on this breaking time are discussed as follows. All the experiments were done on Alginate-Cu²⁺ microcapsules 100. First, the applied voltage was varied. FIG. 6A reveals a monotonic decrease in breaking time with increasing DC voltage. No rupture of the capsules was observed below three volts (3 V) (field strength of one and fourth fifths volts per centimeter: 1.8 V/cm). The breaking time was approximately six minutes (˜6 min) at three volts (3 V), approximately one hundred fifty seconds (˜150 s) at five volts (5 V) and it dropped to approximately seventy seconds (˜70 s) for fifteen volts (15 V). Most of the data collected during capsule breaking was collected at fifteen volts (15 V) (field strength of eight volts per centimeter: 8 V/cm) for convenience. We also studied the capsules under a sinusoidal (AC) field with an amplitude of fifteen volts (15 V) and a frequency of fifty hertz (50 Hz). No swelling or rupture of the capsules was observed in this case.

Next, we varied the parameters influencing the structure of Alginate-Cu²⁺ capsules 100. These capsules are prepared by dropwise addition of two percent (2%) Alginate 104 into a solution of Cu²⁺ 102 (typically 8%). As a liquid drop stays in the Cu²⁺ solution 102, the liquid drop gets converted into a solid capsule. The time the drop remains in the Cu²⁺ solution 102 before being washed is the incubation time (typically, this is held at two minutes: 2 min). The longer this time, the higher the density of crosslinks between Alginate chains 104 and Cu²⁺ ions 102, and thus the stronger the capsule. Also, with a longer incubation time, the crosslinking will be more uniform throughout the droplet, resulting in less variation between the capsule core and shell. FIG. 6B plots the breaking time of capsules under a fifteen volts (15 V) field as a function of the incubation time in an eight percent (8%) Cu²⁺ solution 102. The longer the incubation time, the higher the breaking time. Thus, stronger capsules take longer to break. The Cu²⁺ concentration was varied in the solution from one to sixteen weight percent (1 to 16 wt %), with the incubation time fixed at two minutes (2 min). The breaking time under a fifteen volts (15 V) field increases with increasing Cu²⁺ concentration (FIG. 6C). Increasing the Cu²⁺ is expected to add crosslinks and thus make the capsules stronger, which is again consistent with their taking longer to break.

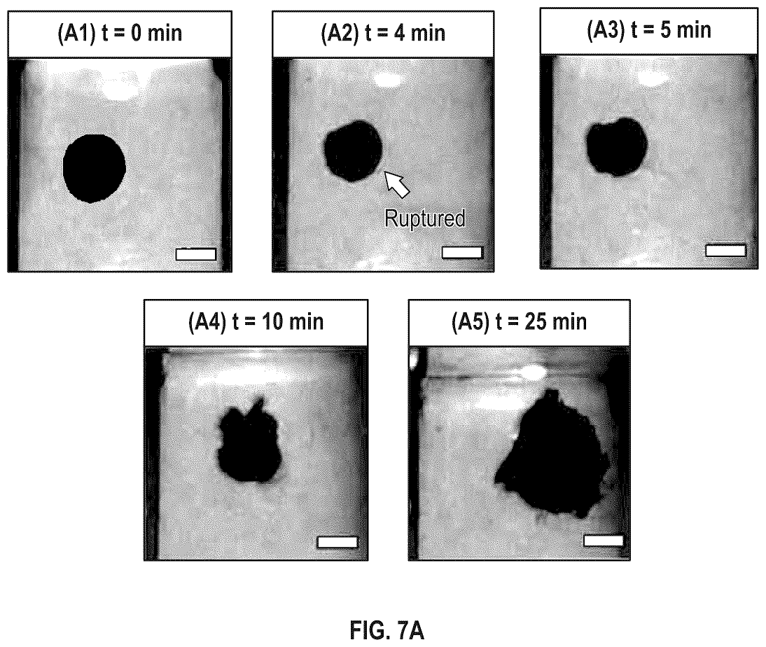

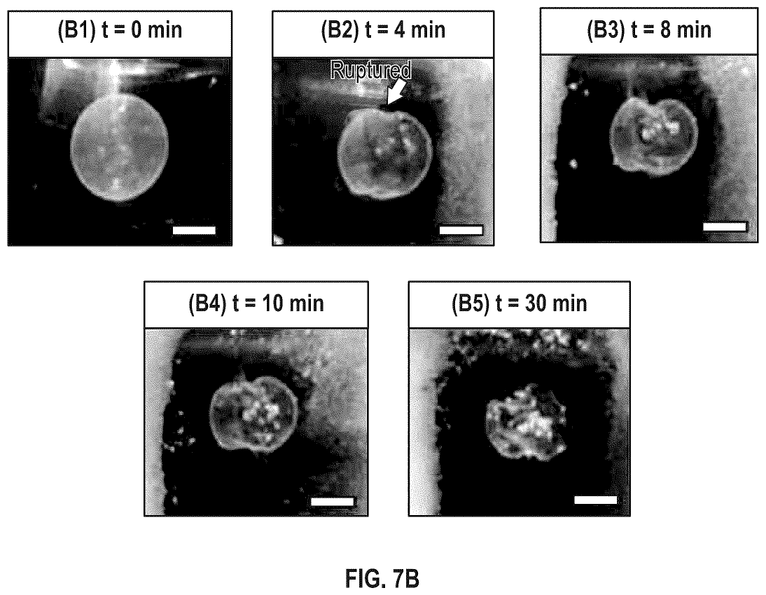

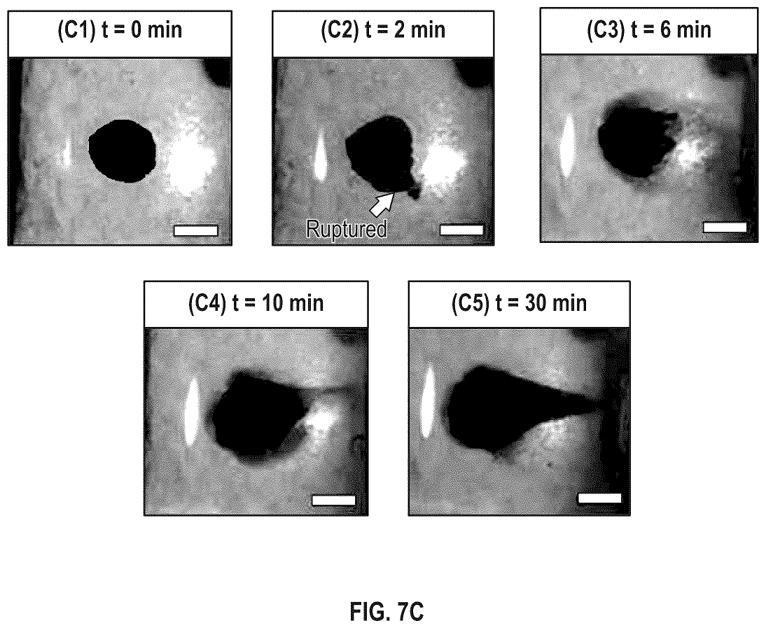

Another variable is the capsule size, which as mentioned above can be varied from the micro to the macroscale. FIGS. 7A-7C shows the electrical response exhibited by macroscale capsules 100, 200, 300 of Alginate-Cu²⁺ (FIG. 7A), Chitosan-SDBS (FIG. 7B), and Gellan-Chitosan (FIG. 7C), respectively. All the capsules have radii around two millimeters (2 mm), which is ˜ten times (10×) larger than their microscale counterparts. For the experiments, the capsules were placed in a ten millimolar (10 mM) NaCl solution and fifteen volts (15 V) was applied. All the capsules break due to the electric field, similar to their microscale counterparts. The Alginate-Cu²⁺ macrocapsules 100 first swell and then break on their side near the + electrode (FIG. 7A), similar to the corresponding microcapsules (FIG. 3B) and in approximately the same time. The Gellan-Chitosan macrocapsules 300 (FIG. 7C) break in a similar manner to their microscale versions (FIG. 4A), but they take longer to do so. But none of the macrocapsules completely disintegrate even after prolonged time in the electric field, which is a difference from the behavior of the microcapsules.

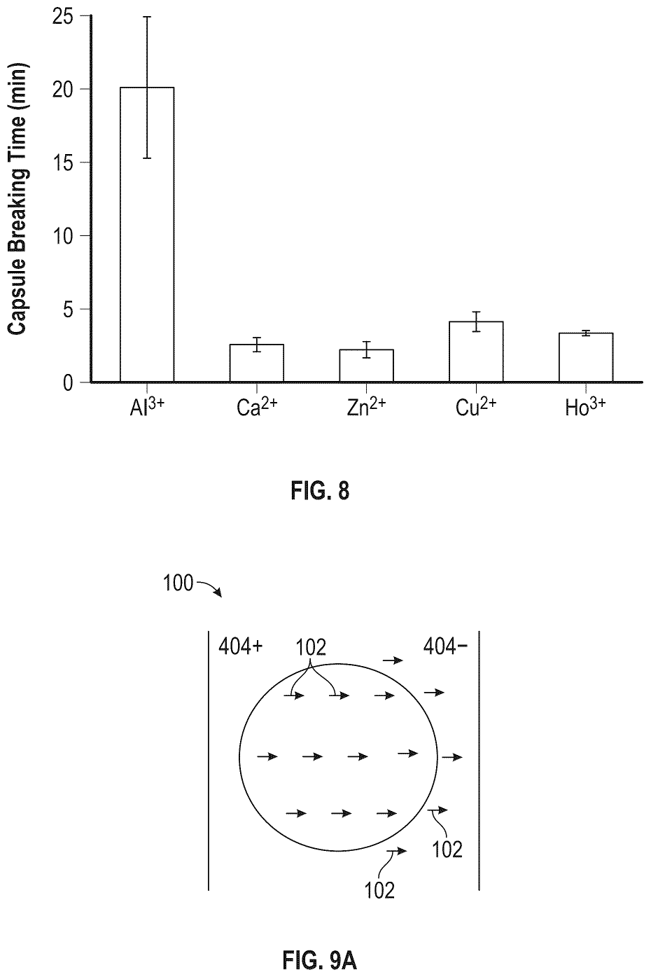

Alginate capsules 100 can be formed by crosslinking with several multivalent cations 102, and cation type was the next variable we studied. We tested macroscale Alginate capsules 100 formed using Cu²⁺ (our typical case), as well as calcium (Ca²⁺), zinc (Zn²⁺), iron (Fe²⁺), aluminum (Al³⁺) and holmium (Ho³⁺). All capsules were formed by dropping two percent (2%) Alginate 104 into eight percent (8%) solutions of the respective cations 102 and incubating for two minutes (2 min). The breaking time of each capsule under a fifteen volts (15 V) field is plotted in FIG. 8. Capsules with divalent cations as the crosslinkers (Cu²⁺, Ca²⁺, Zn²⁺) all broke within five minutes (5 min). In the case of trivalent cations, capsules crosslinked with Ho³⁺ broke within five minutes (5 min) while Al³⁺-crosslinked ones took more than ten minutes (10 min) to break. However, Fe³⁺-crosslinked capsules did not break even after one hour (1 h) under the field. Thus, cation type significantly influences the electrical response of Alginate capsules 100.

All the experiments reported thus far have been done with ten millimolar (10 mM) NaCl as the background electrolyte. The breaking of Alginate-Cu²⁺ capsules 100 in the absence of salt, i.e., in deionized (DI) water, was also examined. In that case, the capsules ruptured when subjected to a voltage of fifteen volts (15 V), but it took longer (greater than fifteen minutes: >15 min) compared to the baseline results. In the absence of salt, the current recorded during the test is very low due to the low ionic conductivity of the solution. One and one hundred millimolar (1 and 100 mM) NaCl was then tried, but there was no significant difference in the breaking time compared to the ten millimolar (10 mM) case. One reason to avoid high NaCl concentrations is because Alginate-Cu²⁺ capsules 100 slowly disintegrate due to exchange of Cu²⁺ with Na+ ions. Based on all these findings, we chose to perform all the other tests with capsules in ten millimolar (10 mM) NaCl solutions.

Determining what the mechanism for electrical disintegration of the capsules causes the capsules to break under an electrical stimulus involves an integration of knowledge from polymer physics, electrochemistry, colloid science, and thermodynamics. It is worth mentioning Alginate capsules 100 can ruptured by an electric field even when embedded in an Agarose gel. Agarose is a nonionic biopolymer that forms gels upon cooling a hot sol. Spherical gels of Agarose (macroscale, approximately two millimeter radius: ˜2 mm) can be made by dropping a hot Agarose solution into a cold reservoir. When these gels are tested in a fifteen volts (15 V) field, they remain intact. This implies that the polymer chains in a capsule or gel must be charged (i.e., should be polyelectrolytes) for electrical rupture to be seen. Capsules formed by contacting Chitosan with glutaraldehyde (GA) were also tested. These capsules do not rupture in a fifteen volts (15 V) field. In these capsules, GA forms covalent bonds between amines on adjacent Chitosan chains. Evidently these covalent bonds are too strong to be broken by an electrical stimulus. Thus, the electrical rupture only occurs in capsules formed by weak, physical bonds of an ionic or electrostatic nature.

Given that polyelectrolytes and electrostatic interactions are present in the capsules 100, 200, 300, pH changes are partially responsible for their specific response. pH dovetails with an electrochemical mechanism. That is, when a current passes through the solution at the voltages studied, water gets electrolyzed, and in turn, a pH gradient is generated in the solution. The pH will be lowered at the anode 404+ due to generation of H+ ions near it and conversely, higher at the cathode 404−. When this pH wave reaches the capsule, and taking the case of an Alginate capsule 100, it was expected the left (+) side of the capsule would experience a lower pH than the right (−) side. The low pH was expected to make the left side shrink. However, surprisingly, the opposite is observed where this side swells before breaking. Also, we studied Alginate-Cu²⁺ capsules 100 in solutions of different pH without an electric field. No changes are seen at high pH, while there was some shrinking of the capsules at low pH. In no case did the capsules break simply due to pH. The distance to the electrode(s) also did not influence capsule breakage (FIG. 5). All in all, an electrochemical mechanism, either due to changes in pH or other electrochemical reactions, cannot explain the breaking of the capsules 100, 200, 300.

The effects of ionic strength and osmotic effects were also considered. As noted earlier, Alginate-Cu²⁺ capsules 100 show electrical rupture regardless of the salt (NaCl) concentration. For comparison, in the absence of the field, if an Alginate capsule 100 is placed in a concentrated (>100 mM) salt solution, the capsule will shrink within a few minutes. If placed back in DI water, the capsule will swell back to its original size. This swelling and shrinking are due to differences in osmotic pressure (i.e., the total concentration of ions and molecular species) between the capsule lumen and the external solution. But these osmotic gradients seem to be insufficient to break the capsules. Overall, if electrical rupture was solely related to osmotic pressure or ionic strength, one would need to explain why these quantities would change sharply upon applying the field. As such, these possibilities can be ruled out as well.

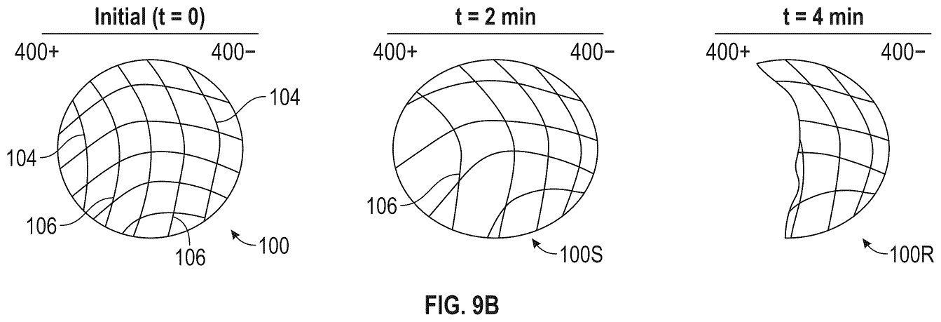

The mechanism for the electrical rupture of the capsules 100, 200, 300 is observed in FIGS. 9A-9B. All the capsules have polyelectrolytes (cationic or anionic) and crosslinking molecules of the opposite charge. With respect to the Alginate capsules 100, anionic Alginate chains 104 are crosslinked by multivalent cations 102 like Cu²⁺ or Ca²⁺. When placed under an electric field, the cations will be pulled by electrostatic forces towards the cathode (−) (i.e., towards the right in FIG. 9A). Equivalently, the Alginate chains 104 will also be pulled towards the anode (+), see e.g., Gargava et al., “Rapid electroformation of biopolymer gels in prescribed shapes and patterns: A simpler alternative to 3-D printing.” ACS Appl. Mater. Interfaces 2019, 11, 37103-37111, hereby incorporated by reference in its entirety herein. Since chains 104 are much larger than the ions 102, their mobility will be nearly negligible. Next, the electrostatic forces on the cations 102 will be sufficient to exceed the strength of their ionic bonds with Alginate chains 104. If so, the cations 102 will be pulled out of their ‘egg-box junctions’ 106 and will then electrophoretically migrate and redistribute themselves within a capsule. This will have a dual effect: on the left (+) side of the capsule 100, the cations 102 will be depleted whereas they will be enriched on the right (−) side. Eventually, some cations 102 may leak out of the capsule 100 at the right edge. The depletion of crosslinks on the left (+) side will first induce that side of the capsule to swell (as shown by the schematic in FIG. 9B), which is what was observed experimentally in FIG. 3. As the electrophoretic migration of cations continues, the removal of crosslinks will eventually cause the capsule to break and disintegrate (FIG. 9C), which is again consistent with the results in FIG. 3.

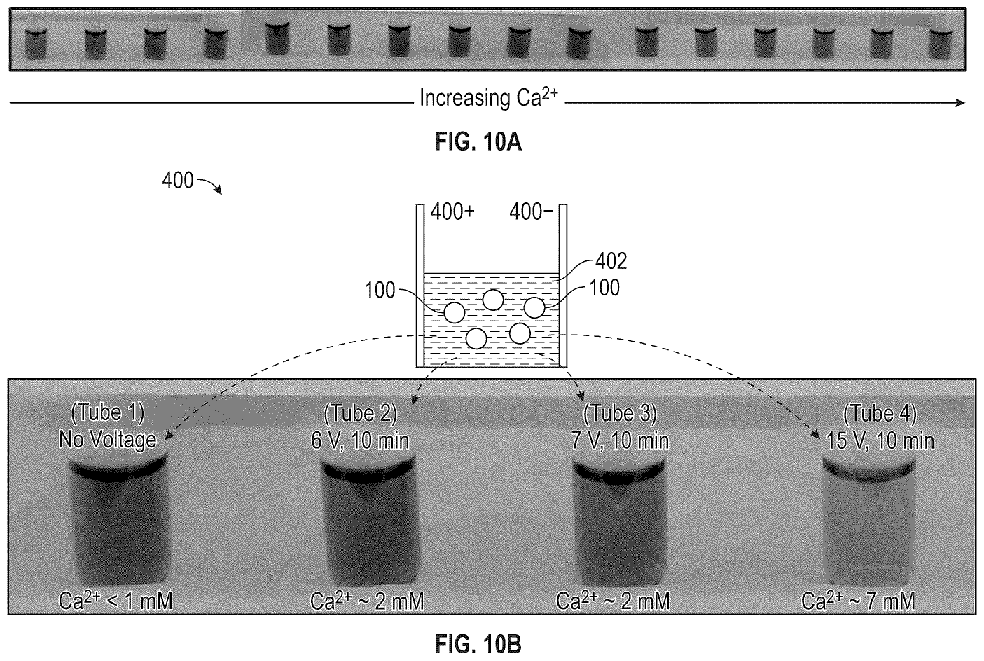

Some cations 102 might escape out of the capsule 100 under an electric field, and this can be tested experimentally. For this, Alginate-Ca²⁺ capsules 100 and eriochrome black T (EBT), a well-known colorimetric indicator for Ca²⁺. An ammonia-buffered solution of EBT is blue, but as Ca²⁺ is added, the solution turns from blue to violet to red (FIG. 10A). Five (5) Alginate-Ca²⁺ macrocapsules 100 were placed in the test cell 400 with two milliliters (2 mL) of ten millimolars (10 mM) NaCl solution (FIG. 10B). The capsules 100 were over-crosslinked by 24 h incubation in Ca²⁺ so that they would not break in the field. Then, the field was switched on at different voltages, after which one milliliter (1 mL) was quickly withdrawn from the test cell 400 (i.e., from around the capsules 100) and mixed with EBT and ammonia (FIG. 1B). In the case of the negative control, i.e., capsules 100 in the test cell for ten minutes (10 min) with no voltage, the EBT solution has a blue color, implying that the external Ca²⁺ is below one millimolar (1 mM) (Tube 1). Next, a voltage of six or seven volts (6 or 7 V) was applied for ten minutes (10 min). The EBT solution now turns from blue to violet (Tubes 2, 3), indicating a slight increase in external Ca²⁺ to approximately two millimolar (˜2 mM). The voltage was then increased to fifteen volts (15 V) for ten minutes (10 min), and in this case, the EBT solution turns light-red (Tube 4), corresponding to approximately seven millimolar (˜7 mM) of external Ca²⁺. The EBT results thus support our hypothesis that the electric field breaks Alginate-Ca²⁺ crosslinks and induces some of the Ca²⁺ to leave the capsules 100 into the external solution. This eventually leads to capsule rupture.

The same mechanism for electrical rupture also applies to Chitosan-SDBS capsules 200 and Gellan-Chitosan capsules 300. In those cases too, the electric field will exert forces in opposite directions on the cationic and anionic species in the capsules. In Chitosan-SDBS capsules 200, the SDBS surfactants 208 are relatively small and comparable to the cations in Alginate capsules 100. In Gellan-Chitosan capsules 300, both the constituents are polymers, but they are expected to be confined to a thin shell. Both these capsules 200, 300 have a liquid core whereas Alginate capsules 100 have a gelled core (FIGS. 1A-1C). Thus, the differences in electrical responses between the three systems studied here can be broadly attributed to: (a) the sizes of their constituents, i.e., polymers vs. small molecules; (b) the strength of interactions between the oppositely charged constituents; and (c) the morphology of the capsules (i.e., whether the core 202, 302 is liquid or gelled and the thickness of the shell 204, 304). Larger molecules will migrate slower under the field, but if they are confined to a thin shell 204, 304, they will only need to be dislodged by a short distance for the capsule to break. More study is needed to further clarify these differences and the relative contributions of (a) to (c). Not all capsules will break under the field, one example being Alginate-Fe³⁺ (FIG. 8).

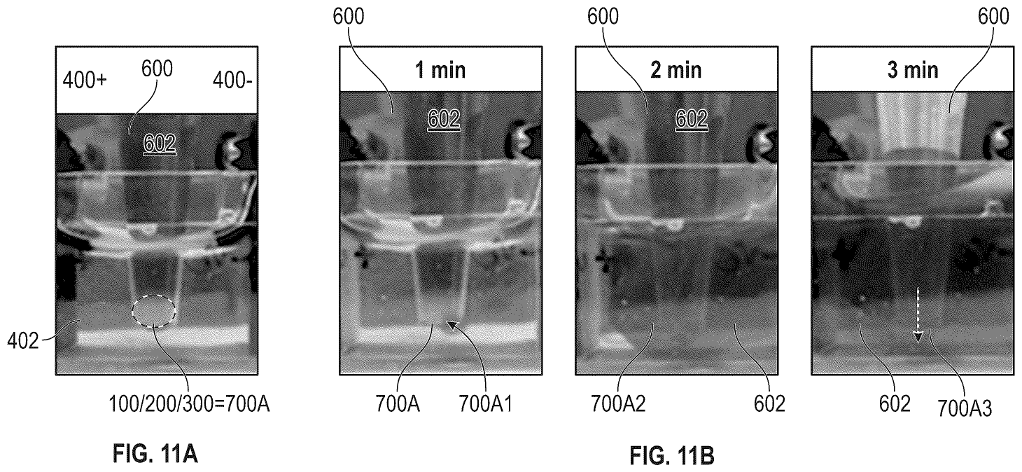

Electrical disintegration can be used to release payloads encapsulated in the capsules such as therapeutics or agrochemicals. Examples of such payload release have been shown in FIGS. 3B, 4B (CB particles) and FIG. 4A (fluorescent particles). Another mode of electrically activated release is demonstrated in FIGS. 11A-11B, and in this case, the capsules 100/200/300 do not have to be destroyed for release to occur. FIG. 11A shows a macroscale Alginate-Cu²⁺ capsule 100 squeezed into the narrow end of a plastic transfer pipette 600. From the other end, the pipette 600 is filled with water containing a model solute (a pink dye, Rhodamine B). This pipette 600 assembly is then placed between electrodes in the test cell, with ten millimolar (10 mM) NaCl (colorless solution 602) surrounding the pipette 600. Initially, the capsule blocks the downward flow of the pink fluid, i.e., the ‘valve’ 700A remains closed. When fifteen volts (15 V) is applied, within about three minutes (3 min), the capsule deforms and breaks (FIG. 11B). Thereby, the valve 700A opens, releasing the pink fluid into the cell, where it mixes with the colorless NaCl solution 602. A key point is that if the capsule is not squeezed tightly in the pipette 600, it will still block the flow—then, even a slight deformation of the capsule by the field will dislodge the capsule and thereby open the valve 700A. If the field is switched off at this stage, the capsule 100/200/300 would be intact, but the actuation of the valve 700A would still have occurred.

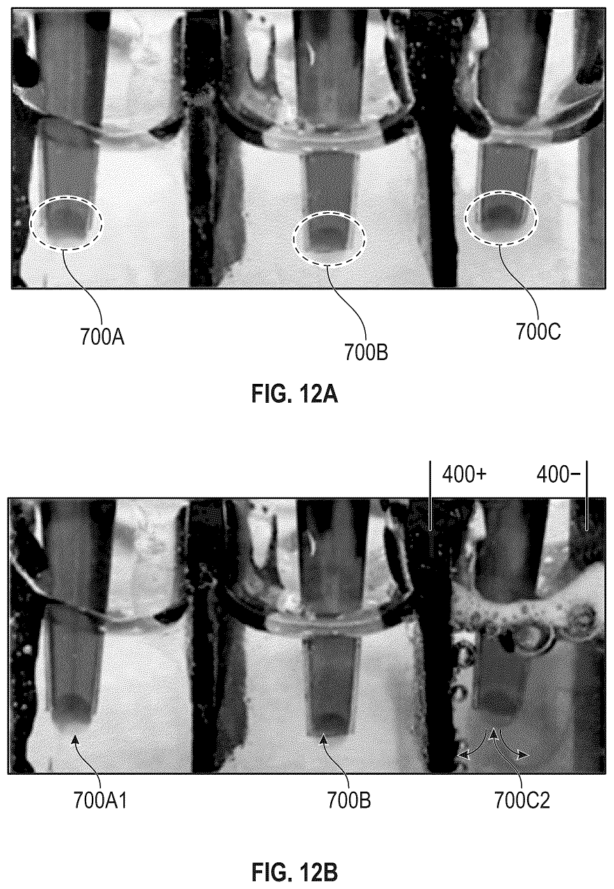

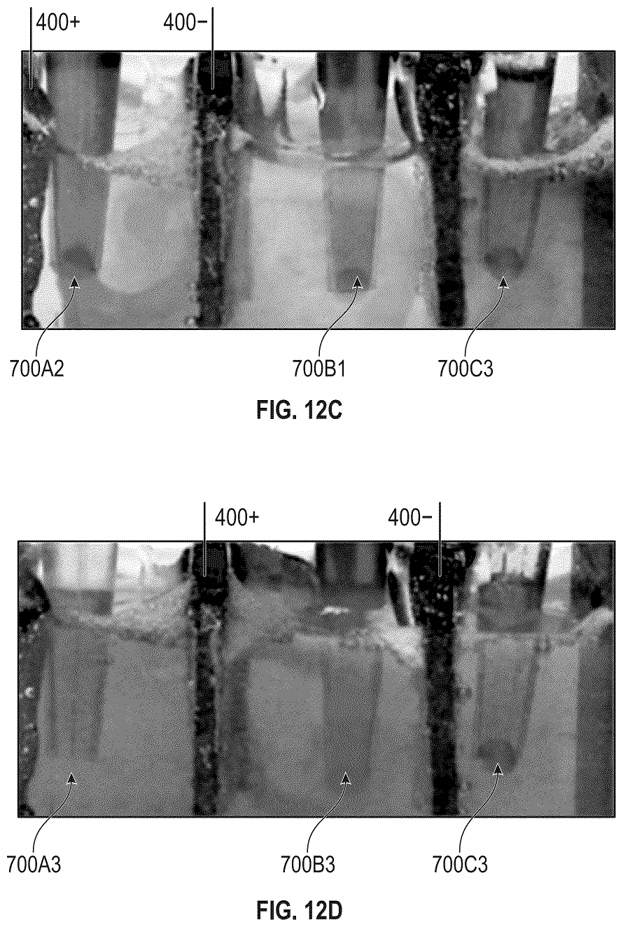

We extended this electro-actuated valve design to incorporate three valves (FIG. 12A). Each valve 700A, 700B, 700C features a pipette 600 bearing a red fluid 602 (Rhodamine 6G in water), with its narrow end blocked by an Alginate capsule 100. The three pipettes are placed next to each other in the same water-filled cell 400, and each pipette 600 is flanked by a pair of graphite electrodes 404+, 404−. This design allows us to actuate each valve 700A, 700B, 700C independently. Initially, all three valves 700A, 700B, 700C are closed, and the fluid in the cell 400 is colorless (FIG. 12B). We first apply a voltage of fifteen volts (15 V) across Valve 3 700C (FIG. 12C) by biasing the two electrodes 404+, 404− on either side of the pipette. This dislodges the capsule and thus opens the valve, causing the red fluid to flow down into the collecting cell 400. Valves 1 and 2 700A, 700B are still closed at this stage because there is no voltage across them. Next, we apply fifteen volts (15 V) across Valve 1 700A, which opens this valve and releases the red fluid contained in it. Valve 2 700B is still closed at this point. Finally, we apply fifteen volts (15 V) across Valve 2 700B and induce this valve to also open. The above sequential opening of valves 700A, 700B, 700C demonstrates several features that could be useful for applications such as drug delivery. For example, the valves 700A, 700B, 700C can also be integrated into remote-controllable devices that are made entirely from soft, biocompatible materials. Such devices ensure that release of drugs or other active ingredients can be switched on at a desired time and place. In yet another example, the three valves 700A, 700B, 700C can correspond to three doses of a drug. One dose can be administered at a time at regular intervals, or if a higher dose is desired, multiple valves 700A, 700B, 700C can be opened at the same time. Also, we can electrically actuate one valve 700A/700B/700C without affecting other ones 700A, 700B, 700C, which is difficult to achieve with other stimuli like temperature and pH. Thus, the setup shows the advantages of an electrical stimulus in terms of spatial precision (ability to be directed at a specific location) and temporal precision (ability to be switched on at a precise instant in time).

### Examples

Materials. Most of the chemicals described above were purchased from Sigma-Aldrich. This included three biopolymers: Alginate (medium viscosity alginic acid, sodium salt from brown algae), Chitosan (medium molecular weight), and Agarose (type I-A, low EEO); and the salts: copper chloride (CuCl₂·2H₂O), calcium chloride (CaCl₂·2H₂O), aluminum sulfate (Al₂(SO₄)₃·xH₂O), zinc sulfate (ZnCl₂·7H₂O), holmium chloride (HoCl₃·7H₂O), and sodium tripolyphosphate (TPP). Other chemicals included sodium hydroxide (NaOH, in pellet form), glutaraldehyde (GA, 50% in water), and the dyes Eriochrome Black T (EBT), Rhodamine B (RB) and Rhodamine 6G (R6G). Iron chloride (FeCl₃, anhydrous) was purchased from Acros Organics, acetic acid (CH₃COOH, glacial) from Fisher Scientific, sodium chloride (NaCl) from EMD Millipore, hydrochloric acid (HCl) from BDH, and ammonium hydroxide from J. T. Baker. The surfactant sodium dodecylbenzenesulfonate (SDBS, hard type) was from TCI America, while the biopolymer Gellan gum 308 (Kelcogel F) was from CP Kelco. Graphite sheets (3 mm thickness) were from Saturn Industries. Carbon black (CB) nanoparticles (N110) were from Sid Richardson Carbon Company. Green-fluorescent polystyrene latex (GFPL) nanospheres (diameter ˜100 nm) were from Polysciences. Deionized (DI) water was used to prepare aqueous solutions.

Macrocapsule Synthesis. To prepare Alginate capsules 100, a feed solution of two percent (2%) of Alginate 104 in deionized (DI) water was dropped into a reservoir solution containing multivalent cations 102, with a typical solution being eight percent (8%) CuCl₂ 102. The incubation time in the reservoir was typically two minutes (2 min). After this time, the capsules were removed, washed with DI water, and stored in a ten millimolar (10 mM) NaCl solution or in DI water. To prepare Chitosan capsules 200, 300, a feed of two percent (2%) Chitosan 206 in 0.2 M acetic acid was dropped into a reservoir solution of five percent (5%) SDBS 208, where it was incubated for three to five minutes (3 to 5 min), then washed and stored as above. To prepare Gellan-Chitosan capsules 300, a feed of one percent (1%) Gellan gum 308 in DI water was dropped into a reservoir of 1% Chitosan 306 in acetic acid. After a three minutes (3 min) incubation, the capsules were washed and stored as above. In all the above cases, the size of the capsules was dictated by the size of the feed droplet, which was varied by using either plastic transfer pipettes or syringe needles of different gauges. A typical radius of each of the above macrocapsules was two centimeters (2 cm). To make capsules containing particles, one quarter percent to one half percent (0.25 to 0.5%) CB or one tenth percent (0.1%) of GFPL were added to the biopolymer feed solution. This was then sonicated using a tip sonicator for one minute (1 min) to disperse the particles prior to its use for capsule synthesis.

In addition to the above capsules, all of which are electroresponsive (FIG. 7A), two other particles were studied as non-responsive controls. Spherical Agarose gels were made by dissolving two percent (2%) Agarose in DI water at ninety degrees Celsius (90° C.) and then adding this hot solution into spherical molds (5 mm radius), followed by cooling to room temperature. Also, Chitosan-GA capsules 300 were made in a manner similar to the Chitosan-SDBS capsules 200 above, with two percent (2%) Chitosan 206 dropped into a mixture of one percent (1%) TPP and (2%) GA and incubated for fifteen minutes (15 min).

Microcapsule Synthesis. Microcapsules (sizes <1 mm) were prepared using a microfluidic method developed by our group that has been described in detail previously. The feed and reservoir solutions for each type of capsule were identical to those mentioned above. The feed flow was controlled by a syringe pump and the feed was sent through a glass capillary tube with an inner diameter typically of 200 μm. Compressed nitrogen gas was sent as a sheath around the capillary. A gas-flow controller was connected to a function generator (BK Precision) to generate gas pulses, with the gas pressure set at fourteen pounds per square inch (14 psi). Details of the setup, together with photos, are provided in the SI section of our earlier paper. For every pulse of gas, an aqueous droplet was dislodged from the tip of the capillary. The flow rate of the liquid as well as the frequency of the pulsing gas dictated the volume of the liquid droplet. Droplets generated this way were very uniform, with polydispersities of <3% in their size. Upon incubation in the reservoir solution, the droplets were converted to microcapsules. Thereafter, they were filtered out, washed with DI water and stored in a ten millimolar (10 mM) NaCl solution.

Electrical Rupture Tests. FIG. 2 shows a schematic and photo of the test cell 400. It includes a transparent cubical box (˜2×2×2 cm) made from plastic, with two planar graphite electrodes fixed vertically in the box on either end and separated by 1.7 cm. The test cell 400 was filled with three milliliters (3 mL) of solution 402, which was typically ten millimolar (10 mM) NaCl. A DC power source (Agilent E3612A) was connected to the two electrodes and a bias of fifteen volts (15 V) was applied across the test cell 400. For monitoring the response of microcapsules, the box was placed on an inverted microscope 500 for observation.

Optical and Fluorescence Microscopy. Brightfield images of the microcapsules under the field were obtained using an inverted optical microscope 500 (Zeiss Axiovert 135 TV) using a two and a half times (2.5×) objective. Fluorescence images of capsules containing GFPL particles were taken using a band pass excitation filter (450-490 nm) and a band pass emission filter (515-565 nm). All images were analyzed using ImageJ software.

Ca²⁺-EBT Colorimetry. The EBT solution was made by dissolving 0.005 g EBT in twenty grams (20 g) DI water. The ammonia buffer was prepared by mixing two milliliters (2 mL) of twenty nine percent (29%) ammonium hydroxide, one milliliter (1 mL) of concentrated HCl, and two milliliter (2 mL) of DI water. Alginate-Ca²⁺ macrocapsules were made as described above with their incubation time extended to twenty four hours (24 h) so that they did not break in the field. For each experiment, five (5) capsules were placed in the test cell 400 along with two milliliters (2 mL) of ten millimolars (10 mM) NaCl (see schematic in FIG. 11B). A given voltage (0, 6, 7, or 15 V) was then applied for ten minutes (10 min). Next, one milliliter (1 mL) of the solution in the test cell 400 (around the capsules 100) was quickly withdrawn using a micropipette and mixed with 150 μL EBT solution and 150 μL ammonia buffer. The color of this solution was then noted. For comparison, standard solutions with different Ca²⁺ concentrations (0 to 0.1% Ca²⁺ in 0.005% increments) were prepared by combining weighed amounts of CaCl₂·2H₂O with 150 μL EBT solution and 150 μL ammonia buffer. The results are shown in FIGS. 12A-12D.

Statistics. Values of the capsule breaking time shown in FIGS. 6A-6C and FIG. 8 were determined from videos of electrical rupture tests and used without any transformation or normalization. At least three samples were tested for each data point. No outliers were excluded. Mean values are shown in the plots and error bars correspond to standard deviations. Statistics were calculated and plotted using Excel and SigmaPlot.

From the foregoing, it can be seen that the present disclosure accomplishes at least all of the stated objectives.

### LIST OF REFERENCE CHARACTERS

The following table of reference characters and descriptors are not exhaustive, nor limiting, and include reasonable equivalents. If possible, elements identified by a reference character below and/or those elements which are near ubiquitous within the art can replace or supplement any element identified by another reference character.

TABLE 1
List of Reference Characters

| | |
|---|---|
| CBP | carbon black particles |
| GFPL | green-fluorescent polystyrene latex |
| 100 | polymer ion capsule (e.g., Aliginate-Cu²⁺/Ca²⁺) |
| 100D | deformed polymer ion capsule |
| 100S | swollen polymer ion capsule |
| 100R | ruptured polymer ion capsule |
| 100C | collapsed polymer ion capsule |
| 100CB | polymer ion capsule loaded with carbon black particles |
| 100CBD | deformed polymer ion capsule loaded with carbon black particles |
| 100CBS | swollen polymer ion capsule loaded with carbon black particles |
| 100CBR | ruptured polymer ion capsule loaded with carbon black particles |
| 100CBC | collapsed polymer ion capsule loaded with carbon black particles |
| 102 | multivalent cations (e.g., Cu²⁺ or Ca²⁺) |
| 104 | anionic polymer (e.g., Alginate) |
| 106 | egg-box junctions |
| 200 | polymer-surfactant capsule (e.g., Chitosan-SDBS) |
| 202 | inner core |
| 204 | outer shell |
| 206 | cationic polymer (e.g., Chitosan) |
| 208 | anionic surfactant (e.g., SDBS) |
| 300 | polymer-polymer capsule (e.g., Gellan-Chitosan) |
| 302 | inner core |
| 304 | outer shell |
| 306 | cationic polymer (e.g., Chitosan) |
| 308 | anionic polymer (e.g., Gellan gum) |
| 400 | rectangular cell |
| 400− | negative electrode |
| 400+ | positive electrode |
| 402 | aqueous solution (e.g., NaCl solution) |
| 500 | inverted optical microscope (e.g., a fluorescence detector) |
| 600 | pipette |
| 602 | dye solution |
| 700A | first valve (blocked state) |
| 700A1 | first valve (deformed state) |
| 700A2 | first valve (early dislodged state) |
| 700A3 | first valve (fully open dislodged state) |
| 700B | second valve (blocked state) |
| 700B1 | second valve (deformed state) |
| 700B2 | second valve (early dislodged state) |
| 700B3 | second valve (fully open dislodged state) |
| 700C | third valve (blocked state) |
| 700C1 | third valve (deformed state) |
| 700C2 | third valve (early dislodged state) |
| 700C3 | third valve (fully open dislodged state) |

### Glossary

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

A “biopolymer” is a natural polymer produced by cells of living organisms.

Biopolymers can comprise monomeric units that are covalently bonded in chains to form larger molecules. Example biopolymers include polynucleotides, polypeptides, and polysaccharides. RNA and DNA are long polymers of nucleotides. Polypeptides include proteins and shorter polymers of amino acids; some major examples include collagen, actin, and fibrin. Polysaccharides are linear or branched chains of sugar carbohydrates; examples include starch, cellulose, and Alginate. Other examples of biopolymers include natural rubbers (polymers of isoprene), suberin and lignin (complex polyphenolic polymers), cutin and cutan (complex polymers of long-chain fatty acids), melanin, and polyhydroxyalkanoates (PHAs).

A “polypyrrole” (“PPy”) is an organic polymer obtained by oxidative polymerization of pyrrole. It is a solid with the formula H(C₄H₂NH)ₙH. It is an intrinsically conducting polymer, used in electronics, optical, biological and medical fields.

A “nanocomposite” is a multiphase solid material where one of the phases has one, two or three dimensions of less than one hundred nanometers (100 nm) or structures having nanoscale repeat distances between the different phases that make up the material.

Biological products include a wide range of products such as vaccines, blood and blood components, allergenics, somatic cells, gene therapy, tissues, and recombinant therapeutic proteins. “Biologics” can be composed of sugars, proteins, or nucleic acids or complex combinations of these substances, or may be living entities such as cells and tissues. Biologics are isolated from a variety of natural sources—human, animal, or microorganism—and may be produced by biotechnology methods and other cutting-edge technologies. Gene-based and cellular biologics, for example, often are at the forefront of biomedical research, and may be used to treat a variety of medical conditions for which no other treatments are available.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

What is claimed is:

1. A system for delivering encapsulated actives comprising:
an electroresponsive biopolymer capsule (100/200/300) comprising:
a cationic component (102/206/306); and
an anionic component (104/208/308) that complements the cationic component;
a cell (400) comprising:
an aqueous solution (402); and
a pair of electrodes (400+, 400−) at least partially submerged in the aqueous solution (402);
wherein electrolysis of the aqueous solution (402) causes an electrophoretic rearrangement of ions or polyelectrolyte chains in the electroresponsive biopolymer capsule (100/200/300), thereby deforming the electroresponsive biopolymer capsule (100/200/300).

2. The system of claim 1, wherein the electroresponsive biopolymer capsule comprises a polymer ion capsule (100).

3. The system of claim 2, wherein the cationic component comprises Cu²⁺ or Ca²⁺ multivalent cations (102), and optionally contained within an 8% CuCl₂ solution.

4. The system of claim 2, wherein the anionic component comprises an Alginate (104), optionally in the amount of 2%.

5. The system of claim 2, wherein the polymer ion capsule (100) is embedded in an Agarose gel.

6. The system of claim 1, wherein the electroresponsive biopolymer capsule comprises an inner core (202/302) and an outer shell (204/304).

7. The system of claim 6, wherein the electroresponsive biopolymer capsule comprises a polymer-surfactant capsule (200).

8. The system of claim 7, wherein the cationic component comprises a Chitosan biopolymer (206), optionally in the amount of 2%.

9. The system of claim 7, wherein the anionic component comprises a sodium dodecyl benzene sulfonate (SDBS) surfactant (208), optionally in the amount of 5%.

10. The system of claim 6, wherein the electroresponsive biopolymer capsule comprises a polymer-polymer capsule (300).

11. The system of claim 10, wherein the cationic component comprises a Chitosan biopolymer (306), optionally in the amount of 1%.

12. The system of claim 10, wherein the anionic component comprises a nonconducting biopolymer including a Gellan gum (308), optionally in the amount of 1%.

13. The system of claim 1, wherein the electroresponsive biopolymer capsule further comprises carbon black (CB) particles or fluorescent polystyrene latex particles.

14. The system of claim 13, further comprising an inverted optical microscope (500) that detects fluorescence.

15. The system of claim 1, further comprising a plurality of electroresponsive biopolymer capsules (100/200/300) that are configured to function as a plurality of independently actuatable valves.

16. The system of claim 1, wherein the aqueous solution (402) is a NaCl solution.

17. An electroresponsive biopolymer capsule (100/200/300) comprising:
a cationic component (102/206/306);
an anionic component (104/208/308) that complements the cationic component; and
an electrophoretic rearrangement of ions or polyelectrolyte chains that when electrically actuated causes the electroresponsive biopolymer capsule (100/200/300) to rupture.

18. A method for delivering encapsulated actives comprising:
loading an electroresponsive biopolymer capsule (100/200/300) with encapsulated actives; and
rupturing the electroresponsive biopolymer capsule (100/200/300) as a result of a applying a direct current (DC) electric field to an electrophoretic rearrangement of ions or polyelectrolyte chains in the electroresponsive biopolymer capsule (100/200/300), thereby causing the encapsulated actives to be released from the electroresponsive biopolymer capsule (100/200/300).

19. The method of claim 18, wherein the rupturing of the electroresponsive biopolymer capsule (100/200/300) occurs regardless of whether there is a change in temperature in the system.

20. The method of claim 18, wherein the rupturing of the electroresponsive biopolymer capsule (100/200/300) occurs without directly contacting the capsule.
